(19) United States
(12) Plant Patent Application Publication
(10) Pub. No.: US 2015/0313070 P1
(43) Pub. Date: Oct. 29, 2015

(54) Gentiana plant named 'Genfire'

(50) ;

(71) Applicant: DALINA GENETICS A/S, Odense N. (DK)

(72) Inventor: Rune Harboe Nielsen, Odense N (DK)

(73) Assignee: DALINA GENETICS A/S, Odense N. (DK)

(21) Appl. No.: 14/120,132

(22) Filed: Apr. 29, 2014

(51) Int. Cl. A01H 5/00

(52) U.S. Cl. PLT/433

(57) ABSTRACT

A new distinct cultivar of Gentiana plant named ‘GENFIRE’, characterized by its globular habit with many branches; thick, ovate dark green leaves and thick, upright buds; and single, campanulate violet-blue flowers.

### LATIN NAME OF THE GENUS AND SPECIES OF THE CLAIMED PLANT

Gentiana scabra

### VARIETY DENOMINATION

‘GENFIRE’

### BACKGROUND OF THE INVENTION

The present invention relates to a new and distinct cultivar of Gentiana, botanically known as Gentiana scabra of the Gentianaceae family, commonly known as Japanese gentian, and hereinafter referred to by the cultivar name ‘GENFIRE’.

The new Gentiana cultivar is a product of a planned breeding program conducted by the inventor, Rune Harboe Nielsen, in Odense, Denmark. The objective of the breeding program is to develop a new Gentiana variety with a good “gentian blue” flower, combined with a good growing character, with many branches and flowers.

The new Gentiana cultivar originated from a cross made in a controlled breeding program by the inventor in June 2008. The female parent is a blue Gentiana scabra variety designated 5797B (not patented). The male pollen parent is a purple Gentiana variety designated 5864H (not patented).

The new Gentiana ‘GENFIRE’ was discovered and selected by the inventor, Rune Nielsen, as a single flowering seedling July 2009 in a controlled environment in Odense, Denmark. Asexual reproduction of the new Gentiana by tissue culture was first performed in January 2010 in Stige, Denmark, and has demonstrated that the combination of characteristics as herein disclosed for the new cultivar are firmly fixed and retained through successive generations of asexual reproduction. The new cultivar reproduces true to type.

### BRIEF SUMMARY OF THE INVENTION

The following traits have been repeatedly observed and are determined to be unique characteristics of ‘GENFIRE’, which in combination distinguish this Gentiana as a new and distinct cultivar:

1. Globular shaped plant; with many branches;

2. Thick ovate dark green leaves. Color of mature leaves RHS Grayed-Green N 189—but greener (upper side), RHS Yellow-green 146B (under side);

3. Thick upright buds, first RHS Yellow Green 144A, transforming into Brown RHS N200 during development and eventually transforming into RHS Grayed-Purple N187B; buds reach a length of 46 mm and a diameter of 15 mm before opening;

4. Single, bell-shaped tubular flowers with basally fused petals, upper third of the inner side of the flower is Violet-Blue N96A, with Violet-Blue 93B edges and small stains of Yellow-Green 144A. Lower 70% of the inside of the tube; the color is White 155C, with small stains of very dark grayed-green to Grayed-Green 189A;

5. Outside of the flower is divided in 2 different colors; the “Bud surface” part of petal is RHS Grayed-Purple N187B, with small tips of Yellow-Green 148A; the “Unfold part” of petal is RHS Violet-Blue N89C;

6. The flowers are 45-55 mm (1.8-2.2 in) long, 35-40 (1.4-1.6 in) mm in diameter, have 5 or 6 lobes and 5 sepals.

The closest commercial variety to the claimed new variety of Gentiana ‘GENFIRE’ is the Gentiana variety ‘GENET’ (Unpatented in the US; registered as CPVO Grant No. 29153). The varieties differ as described in Table 1.

TABLE 1
Comparison with similar variety

| | Comparison cultivar | New Cultivar |
|---|---|---|
| Trait | ‘GENET’ | ‘GENFIRE’ |
| Plant Size | | |
| Height | About 11 cm | About 12-13 cm |
| Diameter | About 20 cm | About 18-22 cm |
| Foliage | | |
| Length | 30-48 mm (1.2-1.9 in) | 20-38 mm (0.8-1.5 in) |
| Width | 13-15 mm (0.5-0.6 in) | 15-25 mm (0.6-1.0 in) |
| Color upper side | RHS Green 135A | RHS Grayed-Green |
| | | N189 (greener) |
| Color under side | RHS Yellow-Green 146B | RHS Yellow-Green 146B |
| Number of Flowers | About 35-45 | About 50-80 |
| per Plant | | |
| Flower Size | | |
| Depth (hight) | 45 mm (1.8 in) | 45-55 mm (1.8-2.2 in) |
| Diameter | 35-40 mm (1.4-1.6 in) | 35-40 mm (1.4-1.6 in) |
| Mature Petal Color | RHS Violet-Blue 96B, | Upper 30% of petal: |
| (upper 30% of inside | with small stains of | Violet-Blue N96A with |
| surface) | Yellow-Green 144A | Violet-Blue 93B edges |
| | | and small stains of 144A |

Plants of the parent varieties, Gentiana 5797B and Gentiana 5864H, are no longer available for comparison with the claimed variety, Gentiana ‘GENFIRE’.

### BRIEF DESCRIPTION OF THE PHOTOGRAPHS

The accompanying photographs illustrate the overall appearance of the new Gentiana ‘GENFIRE’ showing the colors as true as is reasonably possible with colored reproductions of this type. Colors in the photographs may differ slightly from the color value cited in the detailed botanical description, which accurately describe the color of ‘GENFIRE’.

FIG. 1 shows a side view perspective of a typical flowering plant of ‘GENFIRE’ in a 10 cm pot, at 15 weeks of age after planting.

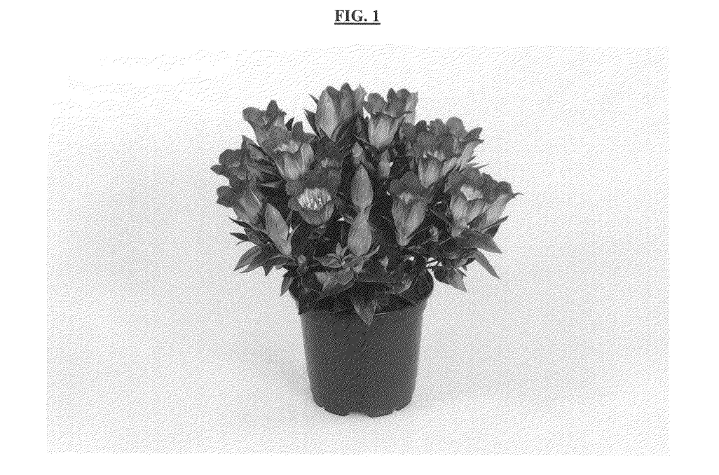

FIG. 2 shows a top view perspective of a typical flowering plant of ‘GENFIRE’ in a 10 cm pot, at 15 weeks of age after planting.

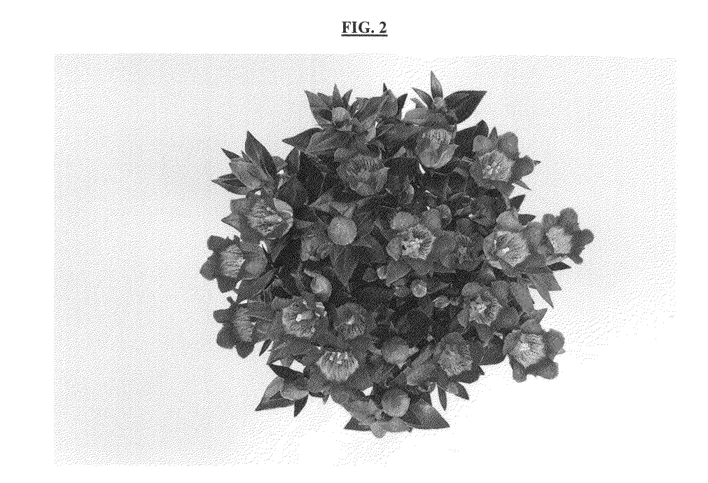

FIG. 3 shows a close-up view of the typical flower of ‘GENFIRE’.

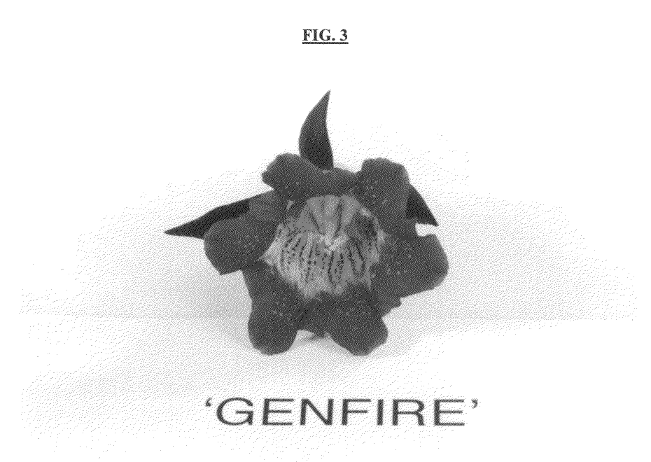

FIG. 4 shows a close-up view from the inside of the typical flowers of ‘GENFIRE’.

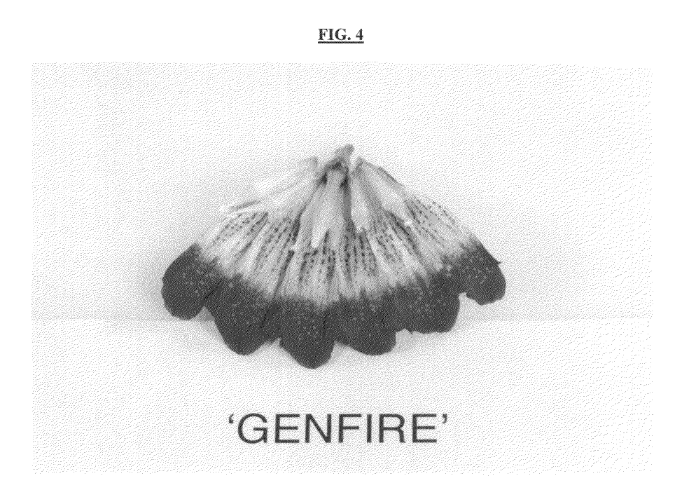

FIG. 5 shows a comparison between the buds and flowers from ‘GENET’ and ‘GENFIRE’.

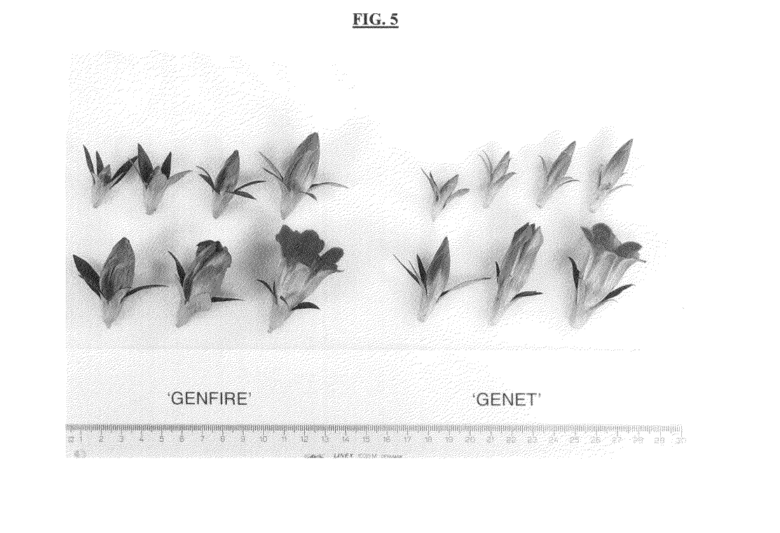

FIG. 6 shows different sizes of leaves from ‘GENFIRE’ at 15 weeks of age after planting.

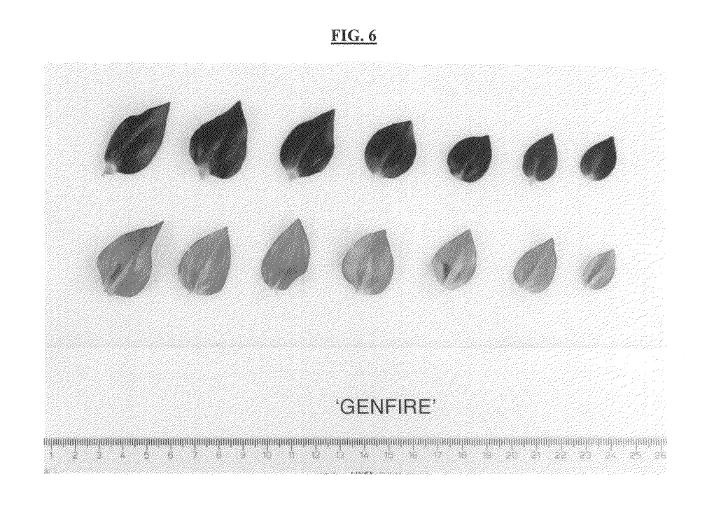

FIG. 7 shows a comparison between leaves from ‘GENET’ and ‘GENFIRE’.

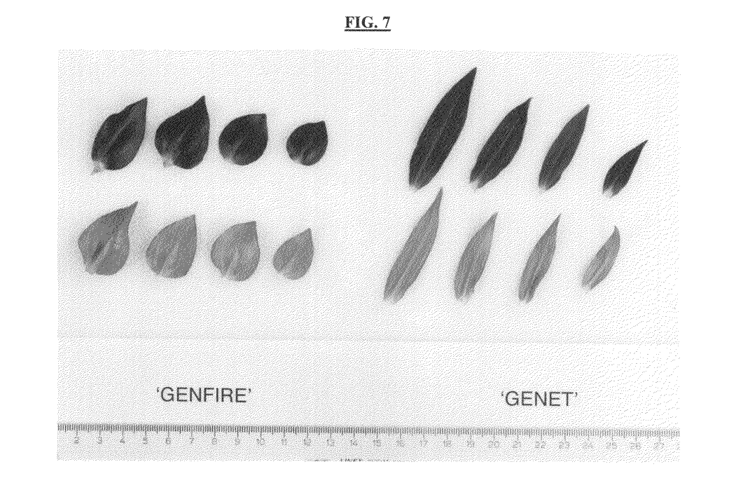

FIG. 8 shows branches with different ages of buds and flowers from ‘GENFIRE’ at 15 weeks of age after planting.

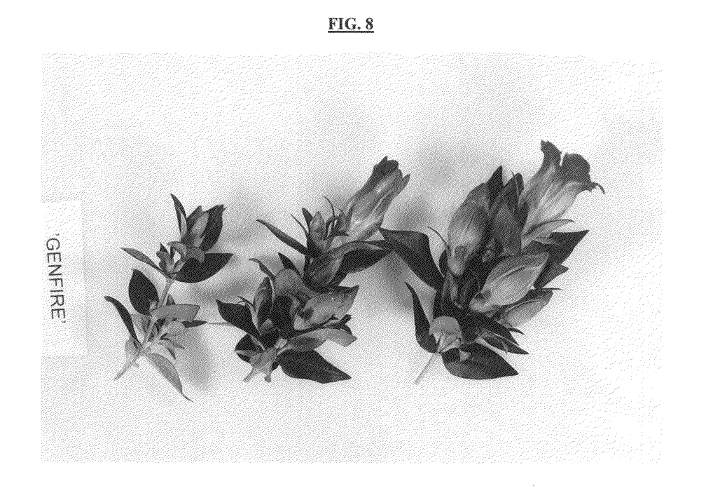

### DETAILED BOTANICAL DESCRIPTION

The new Gentiana ‘GENFIRE’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe the new Gentiana ‘GENFIRE’ as grown in a glasshouse, equipped with heat, assimilation light and ebb/flood watering system in Odense, Denmark under conditions which closely approximate those generally used in commercial practice.

The plants were stuck in paper wrapped propagation pots (3 cuttings per pot), rooted at 22° C. (71.6° F.), and after 5 weeks in propagation, potted into a 10 cm (4 in) pot. After potting they were grown in a greenhouse with long days (>14 hours), heated to 20° C. (68° F.) day and 20° C. (68° F.) night, with a higher temperature during sunny days. The first 5 weeks after potting, the plants were given assimilation light to meet a total of 100 klux per day. From the 6^{th} week until flowering, the plants was given a total of >200 klux per day.

Nutrition was added to the water, and adjusted to keep the EC (μS/M) level in the soil to about 2.0.

The measured plants were not given any chemical growth regulators.

The Color descriptions refer to The Royal Horticultural Society (R.H.S.) Colour Chart 2001 edition. Color values were taken under June daylight conditions, on a day with a thin layer of clouds, around noon in Odense, Denmark.

The age of the GENFIRE plants described is 16 weeks from potting.
Classification:
   Botanical.—Gentiana scabra.
Propagation:
   Type.—Stock plants from tissue culture. Production from top cuttings.
   Time and temperature to initiate roots.—Winter-spring: About 14-16 days at 20-22° C. (68-71.6° F.) in propagation tunnels in a greenhouse.
   Time and temperature to produce a rooted young plant.—Winter-spring: About 35-40 days at 18-20° C. (64.4-68° F.) in a greenhouse.
   Rooting habit.—Fine, fibrous and branching.
Plant description:
   General appearance and form.—Perennial, herbaceous plant with a globular plant habit. Campanulate, tubular flowers arranged in terminal and axillary clusters.
   Growth rate.—Moderately vigorous. Growing about 1-2 cm (0.4-0.8 in) per week until buds are visible.
   Plant height.—About 12-13 cm (4.7-5.1 in).
   Plant width (spread).—About 18-22 cm (7.1-8.7 in).
   Crop time to produce a mature flowering plant.—Propagation period is 5 weeks. After potting, about 15 weeks are required to produce finished flowering plants in 10 cm (4 in) pots.
Stem:
   Shape.—Round.
   Length.—About 7-10 cm (2.8-4.0 in).
   Diameter.—4.0 mm (0.16 in) near the base, and 2.5 mm (0.1 in) under center bud.
   Strength.—Strong.
   Aspect.—Upright to outward.
   Texture.—Hoary with a small, but visible, furrow on either side of stem, and meets the node on the side of the opposite arranged leaves. The furrows rotate 90° on the stem to meet next opposite node on the side.
   Color.—Immature: RHS Yellow-green 145B.
   Mature.—RHS Yellow-green 144A.
   Old.—RHS Yellow-green 151A.
Branches:
   Branching habit.—Basal branching with lateral branching.
   Number of branches per plant.—About 15-20.
   Length (including flowers).—About 6-12 cm (2.4-4.7 in).
   Diameter.—About 1.5-2 mm (0.6-0.8 in).
   Strength.—Strong.
   Aspect.—Bending outwardly in an 30-45° angle from stem.
   Texture.—Hoary with a small, but visible, furrow on either side of stem, and meets the node on the side of the opposite arranged leaves. The furrows rotate 90° on the stem to meet next node on the side.
   Color.—Immature: RHS Yellow-Green 145B.
   Mature.—RHS Yellow-Green 144A.
   Internode length.—About 1.4-1.8 cm (0.6-0.7 in).
   Internode color.—Immature: RHS Yellow-Green 145A.
   Mature.—RHS Yellow-Green 146B.
Foliage description:
   Arrangement.—Opposite, simple, sessile.
   Number of leaves per stem/branch.—About 3-5 pairs per stem/branch.
   Length.—About 20-38 mm (0.8-1.5 in).
   Width.—About 15-25 mm (0.6-1.0 in).
   Overall shape of leaf.—Ovate (W:L aspect ratio=approx. 1:1.5).
   Shape at apex.—Acute.
   Shape at base.—Rounded.
   Margin.—Entire.
   Texture.—Upper surface: Rugose with very fine tesselate pattern. Lower surface: Rugose with very fine tesselate pattern.
   Color of developing foliage.—Upper surface: RHS Green 137A. Lower surface: RHS Yellow-Green 146B.
   Color of mature foliage.—Upper surface: RHS Grayed-Green N 189 (but greener). Lower surface: RHS Yellow-Green 146B.
   Venation pattern.—Parallel.
   Venation color.—Upper surface: RHS Yellow-Green 144A near base, transforming into RHS Grayed-Green N189 (but greener). Lower surface: RHS Yellow-Green 147D near base, transforming into RHS Yellow-Green 146B.
Flower description:
   Appearance.—Terminal and axillary campanulate flowers, arising singularly or in small clusters, held above and beyond the foliage, facing upright or slightly outward.
   Natural flowering season.—Grown outside in Denmark (temperate zone); GENFIRE will flower in late August or early September.
   Time to flower.—140 days from sticking to flowering, when grown in glasshouse environment with heat and assimilation light in Denmark.
   Postproduction longevity.—Flowers maintain good color and substance for 28-35 days on the plant, when grown in an outdoor environment in Denmark. (Longevity of individual flowers is dependent on temperature and light conditions). Inflorescences are persistent.
   Quantity of inflorescences.—Freely flowering; 50-80 buds and open inflorescences per plant.
   Fragrance.—Very insignificant sweet hyacinth-like fragrance.
   Bud.—Rate of opening (from showing color to fully open flower): 5-10 days. Length: About 46 mm (1.8 in) just before opening. Diameter: About 15 mm (0.6 in) just before opening. Aspect: Upright. Shape: Elliptic. Texture: Glabrous. Color: First RHS Yellow-Green 144A, transforming into Brown N200 during development and then transforming into RHS Grayed-Purple N187B before opening. Peduncle: None.
   Flower.—Flower depth (height): 45-55 mm (1.8-2.2 in). Flower diameter: 35-40 mm (1.4-1.6 in). Petals: Quantity per flower: 5 or 6. (the first dominant flowers most often 6; secondary most often 5). Arrangement: Single, bell-shaped, basally fused, tubular flowers. Length: About 35-40 mm (1.4-1.6 in). Width: About 16-20 mm (0.6-0.8 in). Overall shape: Oblong and tubular. Shape at apex: Rounded, irregular. Shape at base: Fused. Margin: Slightly Erose Texture: Upper surface (inner): Glabrous. Lower surface (outer): Glabrous, Striate. Orientation: Initially 20° from vertical, close to 0° from vertical at middle of petal, with development to 180° from vertical at the tip of the petal, forming a tubular flower. Color (when opening): Outer surface: “Bud surface” part of petal: RHS Grayed-Purple N187B with small tips of Yellow-Green 147A; “Unfold” part of petal: RHS Violet-Blue N89C. Inner surface: Upper 30% of petal: Violet-Blue N96A with Violet-Blue 93B edges and small stains of 144A. Lower 70% of petal: RHS 155C with small stains of 189A. Color (when fully opened): Outer surface: “Bud surface” part of petal: RHS Grayed-Purple N187B with small tips of Yellow-Green 148A. “Unfold” part of petal: RHS Violet-Blue N89C. Inner surface: Upper 30% of petal: Violet-Blue N96A with Violet-Blue 93B edges and small stains of Yellow-Green 144A. Lower 70% of petal: RHS White 155C with small stains of RHS Grayed-green 189A. Calyx: Form: Campanulate. Length: About 12-15 mm (0.5-0.6 in). Diameter: About 10-12 mm (0.4-0.5 in). Sepals: Number: 5 (even if there are 6 petals). Length: About 15-25 mm (0.6-1.0). Width: About 5-8 mm (0.2-0.3 in). Overall shape: Lanceolate. Shape at apex: Acute. Shape at base: Fused. Margin: Entire. Texture: Upper surface: Tesselate (very fine). Lower surface: Tesselate (very fine). Color (immature): Upper surface: RHS Green 139A. Lower surface: RHS 144A. Color (when fully opened): Upper surface: RHS Grayed-Green N189A (but greener). Lower surface: RHS 144A. Pedicels: None.
Reproductive organs:
   Androecium.—Stamen number: 5 or 6 (not reflecting number of petals, but 6 stamen are always in 6 petal flowers). Stamen length: About 28 mm (1.1 in). Anther shape: Oblong, almost center fixed. Anther length: About 5 mm (0.2 in). Anther color: RHS Yellow 11C. Filament length: About 11 mm (0.4 in). Filament color: Close to RHS White 155D at base, to Grayed-White 156A near anther. Pollen amount: A lot. Pollen color: RHS Yellow 8B.
   Gynoecium.—Quantity: 1. Pistil length: About 35 mm (1.4 in). Stigma shape: Bifid, curving round when receptive to pollen. Stigma size (receptive surface): Length: About 3.5 mm (0.14 in). Width: 1.5 mm (0.06 in). Stigma color (Receptive surface): RHS White 155C. Style: None. Ovary length: 20 mm (0.8 in). Ovary diameter: (When receptive to pollen) 5 mm (0.2 in). Ovary color: RHS Yellow-Green 145A.
Seed/fruit: Seed and fruit production has not been observed.
Disease/pest resistance: None observed.
Disease/pest susceptibility: Susceptibility to pathogens and pests common to Gentiana has not been observed.
Temperature tolerance: Hardy to USDA Hardiness Zone 7, on soils with good drainage, if well established in the ground prior to the winter.

What is claimed is:

1. A new and distinct cultivar of Gentiana plant named ‘GENFIRE’, as illustrated and described herein.
